(19) United States
(12) Patent Application Publication
(10) Pub. No.: US 2010/0145134 A1
(43) Pub. Date: Jun. 10, 2010

(54) Device for Treatment of Stuttering and Its Use

(75) Inventor: Anja Ravn Madsen, Smorum (DK)

Correspondence Address: BUCHANAN, INGERSOLL & ROONEY PC, POST OFFICE BOX 1404, ALEXANDRIA, VA, 22313-1404, US

(73) Assignee: Oticon A/S, Smorum (DK)

(21) Appl. No.: 12/629,371

(22) Filed: Dec. 2, 2009

(30) Foreign Application Priority Data
Dec. 2, 2008 (EP) 08105915.6

(51) Int. Cl. A61F 5/58

(52) U.S. Cl. 600/23

(57) ABSTRACT

The invention relates to a portable listening system comprising a microphone device for picking up a person's voice and a listening device comprising a receiver. The invention further relates to the use of such system. The object of the present invention is to provide a portable device for treatment of stuttering. The problem is solved in that the system being adapted to pick up the voice of a person wearing the portable system by the microphone device, converting the voice to an own voice electrical signal, to transmit the own voice electrical signal to the listening device, to receive the own voice electrical signal in the listening device and to play it for the person by the receiver with a delay. An advantage of the invention is that it provides a flexible body worn system suitable for treatment of stuttering. The invention may e.g. be used for applications involving listening devices to be worn by a person to be treated for stuttering.

### TECHNICAL FIELD

The present application relates to a device for providing aid against stuttering. The application relates specifically to a portable listening system comprising a microphone device for picking up a person's voice and a listening device comprising a receiver.

The application furthermore relates to use of a portable listening system.

The application may e.g. be useful in applications involving listening devices to be worn by a person to be treated for stuttering.

### BACKGROUND ART

The prevalence of stuttering estimated to be between 0.7 and 1% of the adult population.

Prior art devices that uses delayed sound for treatment of stuttering exist.

There are, however, different problems with these, including:
They are fairly expensive
They are adapted for use only at home (e.g. with headphones, wire and external microphone), not suitable for direct face-to-face communication with others
They don't offer a proper solution, when talking on the phone
They do not provide a general solution for use by a hearing impaired.

It is known in the prior art to use a delay of own voice to influence stuttering. It is also known from WO 2005/037153 A1 to use a bone conducting hearing aid for that purpose. U.S. Pat. No. 5,961,443 describes a device and method for ameliorating stuttering by providing an altered auditory feedback in the form of a portable, self-contained device including a receiver for receiving auditory signals, an auditory delay and or frequency shift circuit, and a transmitter for transmitting the altered signal to the individual user. Other systems providing a masking signal for assisting a user in overcoming stuttering are disclosed. DE 35 06 092 A1, U.S. Pat. No. 4,662,847, DE 31 46 556 A1, WO 81/02513 A1 disclose systems where the masking signal is a delayed version of a users voice. U.S. Pat. No. 3,566,858 discloses a system wherein a frequency variation of the masking signal is provided in accordance with variations of the pitch of a user's voice. WO 94/00085 A1 and WO 2006/060243 A2 disclose systems for digitally processing a microphone signal comprising a user's voice before it is presented to the user, where a different processing is applied for different frequency bands of the input signal.

Further, U.S. Pat. No. 5,794,203 deals with a biofeedback system for speech disorders, which is adapted to detect disfluent speech (e.g. using an electromyograph (EMG)) and to provide auditory feedback enabling immediate fluent speech and to control the auditory feedback in accordance with the disfluent speech to enable immediate carryover fluency. The biofeedback system may also be provided with delayed auditory feedback and may be provided with a telephone interface to enable fluent speech while talking on telephones.

### SUMMARY

An object of the present application is to provide a portable system for treatment of stuttering.

Objects of the application are achieved by the invention described in the accompanying claims and as described in the following.

An object of the application is achieved by a portable listening system comprising a microphone device for picking up a person's voice and a listening device comprising an output transducer (e.g. a receiver). The system is adapted to pick up the voice of a person wearing the portable system (a user of the system) by the microphone device, converting the voice to an own voice electrical signal, to transmit the own voice electrical signal to the listening device, and to receive the own voice electrical signal in the listening device, the system comprising a delay unit allowing to present the own voice signal for the person wearing the portable system (i.e. the user of the system) by the output transducer (e.g. a receiver) with a configurable delay.

An advantage of the invention is that it provides a flexible body worn system suitable for treatment of stuttering.

Preferably, the portable listening system comprises at least one local energy source, such as a battery, e.g. a rechargeable battery. Preferably, all functions of the portable listening system are energized by the local energy source. In an embodiment, the local energy source is the only source of electric energy for the listening system.

If a delay in the signal transmitted from an intermediate body worn communication device comprising the microphone device to the listening device (e.g. a hearing aid) is implemented, the combined system would be ideal to use for stutterers that seek treatment with delayed sound—especially stutterers with hearing loss can benefit from this system.

The combined system will give the wearer (hearing disabled or not) a portable system that can be used whenever wanted or needed—even when talking on the phone or in other situations, where no other solution is offered so far.

A hearing aid is nowadays so small that it is cosmetically to be preferred over e.g. headphones when in dialogue with others. Also, and more importantly, the speech from the wearer can e.g. be picked up and delayed by the intermediate communication device, whereas the speech from the person talking to you can be picked up—and not delayed—by the hearing aid. This will enable the stutterer to use the system in dialogue and in phone conversations with others.

In an embodiment, the microphone device and the listening device constitute two physically separate bodies, each having their own dedicated housing and either being not physically connected or physically connected by a releasable or fixed connecting mechanism, e.g. an electrical (and/or mechanical, e.g. acoustical) connector. In an embodiment, the portable listening system is embodied in a hearing instrument of the BTE-type, e.g. comprising a BTE-part adapted for being located at or behind the ear of a user and an ITE-part (e.g. comprising an output transducer) adapted to be located in the ear canal or in the ear of the user. Alternatively, the microphone and the listening device may form part of the same physical body (e.g. a hearing instrument of the ITE-type adapted to be located completely in the ear canal of a user).

In an embodiment, the system comprises two or more microphones, at least one of which is part of the microphone device for picking up a user's own voice. The microphone for picking up a user's own voice is preferably located at a specific position suitable for such purpose.

In an embodiment, the portable listening system comprises an own voice detector for detecting whether a given input sound (e.g. a voice) originates from the voice of the user of the system. Own voice detection is e.g. dealt with in US 2007/009122 and in WO 2004/077090. In an embodiment, the microphone device is adapted to be able to differentiate between a user's own voice and another person's voice.

In an embodiment, the system is adapted to present the user's voice with a (configurable) delay to the user whereas the voice of another person is not delayed. This can e.g. be achieved based on a control signal from an own voice detector. Alternatively, it can be achieved by using dedicated microphones (or microphone system modes) for each of the user's own voice and another person's voice, respectively. In an embodiment, an own voice detector is used to control the configurable delay.

In an embodiment, the microphone device and the listening device are distinct, physically separate parts that only cooperate electromagnetically via a wireless (e.g. near-field, e.g. inductive, or far-field (radiated)) connection between them. No wired connections (or connectors) exist between the two devices.

In an embodiment, the listening device comprises a (further) microphone for picking up an input sound. In an embodiment, the listening device comprises a microphone, which (e.g. in a specific mode or program of the listening device) is dedicated for picking up another person's voice (other than the user of the system).

In an embodiment, the portable listening system is adapted to provide that the voice of a person with whom the wearer of the portable listening system communicates is picked up by the or a microphone of the listening device. Thereby the wearer's own voice is delayed for the wearer, but the voice or sounds of the environment are not delayed.

In an embodiment, the portable listening system is adapted to provide that the transmission between the microphone device and the listening device is wireless, e.g. based on inductive communication. In an alternative embodiment, the portable listening system is adapted to provide that the transmission between the microphone device and the listening device is wired, e.g. via an electrical connector, e.g. connecting a head mounted microphone with a listening device, e.g. a hearing instrument. Alternatively the microphone device may take the form of a click on-module adapted for being readily electrically connected to a listening device, e.g. via a (e.g. standardized) direct electric input (DEI) input (e.g. like an FM-shoe, cf. e.g. FIG. 1c).

The signal path of the portable listening system from the microphone device to the output transducer has an inherent, minimum delay depending on the application in question (e.g depending on the delay of the components of the signal path). Likewise, the signal path from input to output transducer of a hearing instrument has a certain minimum time delay. In general, the delay of the signal path is adapted to be as small as possible. In the present context, the term ‘delay’ or ‘configurable/adjustable delay’ is taken to mean an additional delay (i.e. in excess of the minimum delay of the signal path) that can be appropriately adapted to the user's needs. Preferably, the configurable delay can be set to a number of distinct values or to values in a continuous range including zero (extra) delay (e.g. between a minimum delay (e.g. 0) and a maximum value (e.g. 1 s)).

In an embodiment, the portable listening system is adapted to provide that the (additional) delay is larger than 30 ms, such as in the range from 50 to 100 ms, such as larger than 100 ms, such as in the range from 100 ms to 500 ms, e.g. between 200 ms and 300 ms, such as larger than 500 ms. In an embodiment, the portable listening system is adapted to provide that the configurable delay can be set continuously or in steps (of e.g. 5 ms or 10 ms), e.g. by a user and/or during fitting, between a minimum value (e.g. 0 ms) and a maximum value (e.g. 100 ms or 500 ms or 1 s).

In an embodiment, the delay is implemented as a buffer, either in hardware or software. In an embodiment an adjustable delay is implemented by having a number of buffers, each having a certain delay, and wherein the circuitry is adapted to be able to lead the (own voice) electrical signal through a configurable number of said buffers to create a certain delay in time of the (own voice) electrical signal (and thus of the acoustical signal presented to the user). In an embodiment, an adjustable delay is implemented as a software buffer, adjustable to a certain delay in steps between a minimum and a maximum value.

In an embodiment, the portable listening system comprises a communication device. In an embodiment, the portable listening system comprises a headset, a hearing instrument, a mobile telephone, an entertainment device (e.g. a music player) or a combination thereof.

In an embodiment, the portable listening system takes the form of a hearing instrument, wherein the microphone device for picking up a person's voice is a microphone of the hearing instrument or form part of the microphone system of the hearing instrument. Preferably, the hearing instrument comprises an own voice detector for detecting the user's own voice and for controlling or influencing the application of the configurable delay.

In an embodiment, the listening device is a hearing instrument. In an embodiment, the listening device is a hearing instrument for processing an input sound to an output sound according to a user's needs, the hearing instrument comprising an input transducer (e.g. a microphone or a microphone system, e.g. an adaptive directional microphone system) for converting an input sound to an electric input signal and an output transducer (e.g. a receiver) for converting a processed electric output signal to an output sound, a forward path being defined between the input transducer and the output transducer, the forward path comprising a signal processing unit adapted for providing a frequency-dependent gain and for providing a processed output signal. In an embodiment, the hearing instrument comprises a cochlear implant. In an embodiment, the hearing instrument comprises a bone conducting device (bone anchored device).

In an embodiment, the portable listening system is adapted to provide that the delay is at least partially (such as that a major part of the delay is) implemented in the microphone device. Alternatively, the delay may be fully or partially implemented in the listening device.

In an embodiment, the microphone device comprises an audio selection device for selecting an audio signal from a multitude of audio signals and forwarding the selected audio signal to the listening device.

In an embodiment, the portable listening system is adapted to provide that a selected audio signal can be a signal from a telephone, e.g. a mobile telephone. Alternatively, the selected signal can be an audio signal from a PC, e.g. in connection with a telephone conversation conducted via the Internet (e.g. as an IP-telephone conversation or as part of a video conference or remote learning programme, such as a web-demo).

In an embodiment, the microphone device comprises a mobile telephone.

In an embodiment, the portable listening system is adapted to provide that an audio signal representing an incoming signal from a telephone, e.g. the voice of another person, is forwarded to the listening device without additional delay.

In an embodiment, the portable listening system is adapted to provide that a user can enable and/or disable the delay of the user's own voice.

In an embodiment, the portable listening system is adapted to provide that a user can enable a delay of an incoming signal from a telephone corresponding to and/or independent of the delay of the own voice signal.

In an embodiment, the portable listening system is adapted to provide that the delay of the user's own voice is automatically enabled when a telephone conversation is initiated. In an embodiment, the delay unit is set to a predefined (e.g. user configurable) delay (e.g. 50 ms) of the users own voice when entering a telephone mode (i.e. when a telephone conversation is initiated, either by the user or by an incoming call).

In an embodiment, a microphone of the listening device is automatically disabled when a telephone conversation is initiated. Alternatively, the portable listening system comprises a manual switch for disabling/enabling a microphone of the listening device and/or for enabling/disabling the delay of the user's own voice.

In an embodiment, the output transducer is a receiver of a normal listening device, or an electrode of a cochlear implant, or a vibrator of a bone conducting device.

Use of a portable listening system as described above, in the section ‘mode(s) for carrying out the invention’, in the drawings and in the claims is furthermore provided by the present invention.

Further objects of the invention are achieved by the embodiments defined in the dependent claims and in the detailed description of the invention.

As used herein, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements maybe present, unless expressly stated otherwise. Furthermore, “connected” or “coupled” as used herein may include wirelessly connected or coupled. As used herein, the term “and/or” includes any and all combinations of one or more of the associated listed items. The steps of any method disclosed herein do not have to be performed in the exact order disclosed, unless expressly stated otherwise.

### BRIEF DESCRIPTION OF DRAWINGS

The disclosure will be explained more fully below in connection with a preferred embodiment and with reference to the drawings in which:

FIG. 1 shows various application setups of embodiments of a portable listening system according to the present application;

FIG. 2 shows various partitions of a portable listening system according to the present application;

FIG. 3 shows an application scenario of an embodiment of a portable listening system according to the present application, and

FIG. 4 shows an embodiment of a portable listening system.

The figures are schematic and simplified for clarity, and they just show details which are essential to the understanding of the disclosure, while other details are left out.

Further scope of applicability of the present application will become apparent from the detailed description given hereinafter. However, it should be understood that the detailed description and specific examples, while indicating preferred embodiments of the application, are given by way of illustration only, since various changes and modifications within the spirit and scope of the invention will become apparent to those skilled in the art from this detailed description.

### MODE(S) FOR CARRYING OUT THE INVENTION

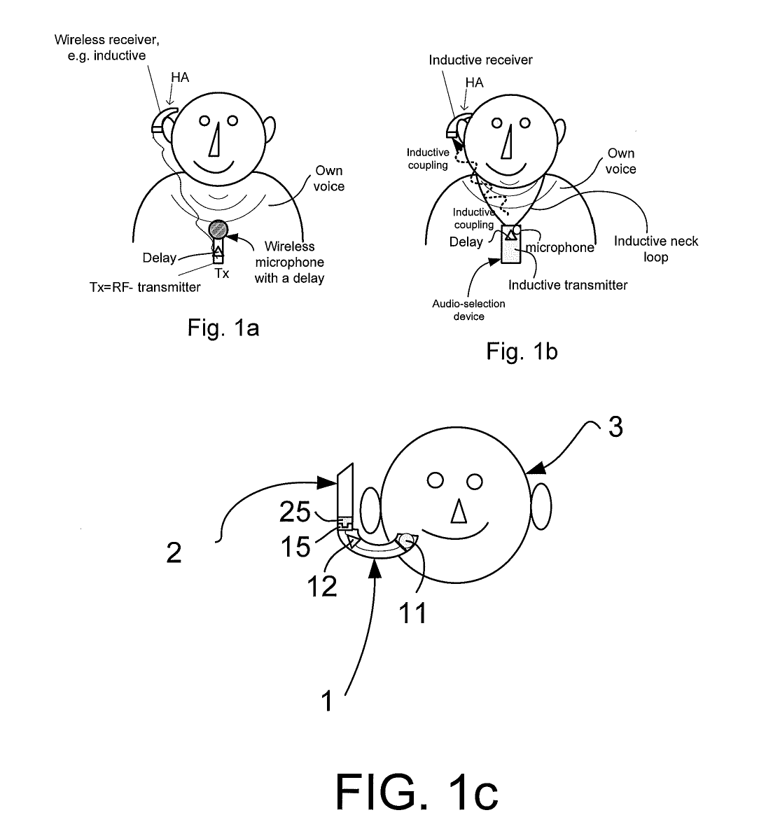

FIG. 1 shows various application setups of embodiments of a portable listening system according to the present application.

FIG. 1a shows an embodiment of a portable listening system comprising a microphone device comprising a wireless microphone for picking up the voice of a person and converting the voice to an own voice electrical signal, a listening device, here in the form of hearing instrument (HA), the wireless microphone comprising a (preferably configurable) delay unit for delaying the own voice electrical signal and a transmitter for wirelessly transmitting a signal comprising the delayed own voice signal to a corresponding receiver in the hearing instrument. The wireless transmission can either be based on radiated fields between respective antennas of the wireless microphone and the hearing instrument or based on inductive coupling between respective induction coils. The wireless transmission can be analogue (e.g. FM) or digital (e.g. frequency hopping spread spectrum). The hearing instrument is adapted to receive the own voice electrical signal, to process it according to a user's hearing profile and to play it for the person by the receiver with a delay. The delay is adapted to be user programmable (e.g. by an adjustable activator, e.g. a turning knob or toggle switch or via a user interface on a display) e.g. in the range from 0 ms to 100 ms, such as from 10 ms to 50 ms.

FIG. 1b shows an embodiment of a portable listening system comprising a communication device, here an audio selection device for selecting an audio signal from a multitude of audio signals and forwarding the selected audio signal to a listening device, here a hearing instrument (HA). The audio selection device comprises a microphone for picking up the voice of the person wearing the portable listening system and for converting the voice to an own voice electrical signal. The audio selection device comprises a delay unit for delaying the own voice electrical signal and a transmitter for wirelessly transmitting a signal comprising the delayed own voice signal to a corresponding receiver in the hearing instrument. In the present embodiment, the wireless transmission is based on inductive coupling between coils in the two devices or between a neck loop antenna distributing the field from a coil in the audio selecting device to the coil of the hearing instrument. Alternatively, the neck loop antenna can be driven directly from the transmitter of the audio selection device. The transmitted signal comprising the audio signal may be the baseband audio signal or a modulated carrier signal (e.g. a carrier modulated with the audio signal) or a digital signal comprising the audio signal.

The audio selection device is adapted to be able to mediate a telephone conversation, i.e. receive an incoming audio signal from a telephone (e.g. a mobile (or cellular) telephone) and to wirelessly forward it to the hearing instrument and to transmit the own voice signal picked up by the microphone of the audio selection device and transmit it to the telephone. The transmission to and from the telephone may e.g. be according to the Bluetooth protocol. In an embodiment, the received audio signal from a telephone is forwarded to the hearing instrument without additional delay, whereas the user's own voice picked up by the microphone of the audio selection device is delayed to counteract the user's stuttering. In an embodiment, user's own voice is NOT delayed when transmitted to the telephone (to be forwarded to a receiver ‘at the other end of the line’ without additional delay). Alternatively, the received audio signal from a telephone is forwarded to the hearing instrument with the same additional delay as that applied to the own voice signal. In an embodiment, the microphone system of the hearing instrument is disabled during a telephone call, when the hearing instrument receives an audio signal from a telephone apparatus.

The wireless microphone of the embodiment of FIG. 1a and the microphone of the embodiment of FIG. 1b are specifically adapted to pick up the voice of the wearer of the portable listening system, e.g. in that it has a directionality that enables a direction towards the mouth of the wearer (when the system is appropriately mounted for normal operation). The portable listening system may comprise a voice detector for detecting whether a microphone signal contains a human voice. Alternatively or additionally, the portable listening system comprises an own voice detector for differentiating the origin of a microphone signal between a user's own voice and other voices or sounds.

FIG. 1c shows an embodiment of a portable listening system comprising a microphone device 1 comprising a microphone 11, here mounted on a longitudinal arm, like a headset microphone. The microphone device is adapted for being positioned close to a users' mouth when the microphone device is mounted (e.g. clicked) on the listening device mounted at the ear of the user 3. The microphone device comprises a delay unit 12 electrically connected to the microphone 11. The delay unit is adapted for adjustably delaying the user's voice as picked up by the microphone. In another embodiment, the delay unit may be located in the listening device. In an embodiment, the user's voice is only delayed when being presented to the user, NOT when being transmitted away from the user, e.g. to a receiving person via a transmission net, e.g. a telephone or data net. In the embodiment of FIG. 1c the connection between the microphone device 1 and the listening device 2 is not wireless, but the transmission from the microphone device 1 to the listening device 2 is conducted in the same way as from an FM shoe to a hearing aid via an electrical connector (comprising two matching electrical connectors 15, 25, e.g. of a plug 25 of the listening device 2 and a socket 15 of the microphone device 1 (or vice versa)). In an embodiment, the portable listening system takes the form of a hearing instrument, wherein the microphone device for picking up a person's voice is a microphone of the hearing instrument. Preferably, the hearing instrument comprises an own voice detector for detecting the user's own voice and for controlling or influencing the application of the configurable delay. Preferably, the hearing instrument comprises an adaptive directionality system for separating a plurality of acoustic sources, and for controlling or influencing the application of the configurable delay. Such systems, e.g. based on beam forming, e.g. using time frequency masking, are used to (dynamically) determine a direction of an acoustic source and/or to segregate several acoustic source signals originating from different directions are e.g. discussed in EP 0 869 697, EP 1 005 783, and [Pedersen et al., 2005].

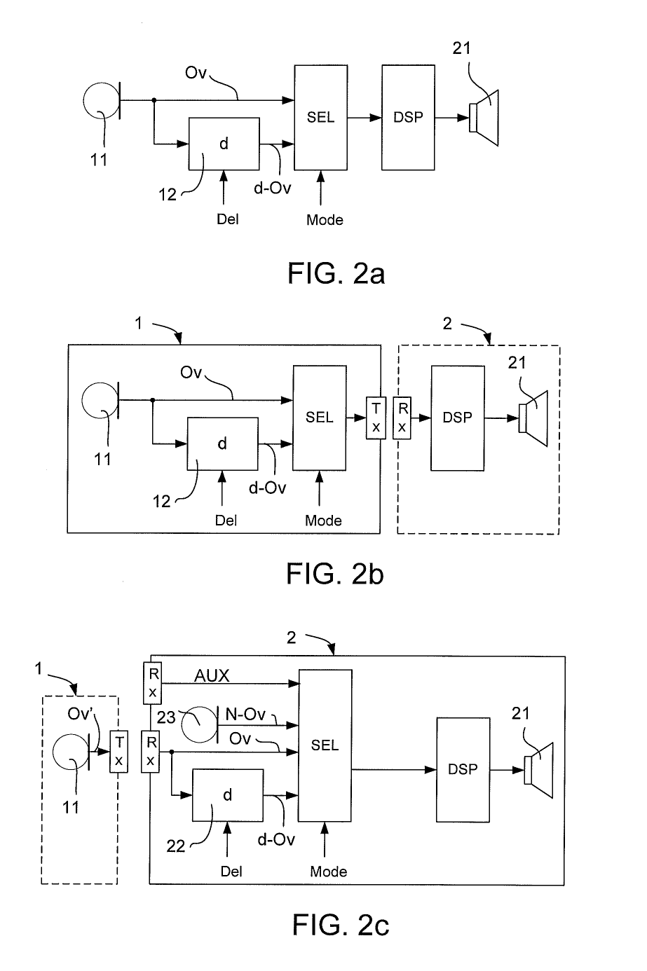

FIG. 2 shows various partitions of a portable listening system according to the present application. The basic components of a portable listening system according to the present application is a microphone device for picking up a person's voice and a listening device for presenting sound to a user as shown in FIG. 2a. The system is adapted to pick up the voice of a person wearing the portable system by the microphone device, converting the voice to an own voice electrical signal, to transmit the own voice electrical signal to the listening device, to receive the own voice electrical signal in the listening device and to play it for the person by the receiver with a delay. In the embodiment of FIG. 2a, no partition between the microphone device (comprising at least the microphone 11) and the listening device (comprising at least the receiver 21) is indicated. The microphone 11 is adapted to convert a person's voice to an electrical own voice signal Ov, which is fed to a delay unit (d) 12 as well as to a selector unit (SEL). The delay unit 12 is adapted to delay the input signal Ov with an adjustable delay based on an input Del and to provide a delayed output signal d-Ov, which is fed to the selector unit (SEL). The selector unit is adapted to select one of its inputs Ov or d-Ov based on a mode input Mode and thus to provide a selected output signal (being either an undelayed or a delayed version of the own voice signal Ov), which (here) is fed to an optional processing unit (DSP) for further signal processing, e.g. for providing a frequency dependent gain, e.g. adapted to a user's needs. The processed output of the signal processing unit (DSP) is fed to the receiver 21 for converting the processed output signal to an output sound for being presented to the user of the portable listening system. The receiver 21 may alternatively be electrodes of a cochlear implant or a vibrator of a bone conducting device. The portable listening system may further comprise other functional blocks, such as an anti feedback system for minimizing the effect of acoustic feedback from the receiver to a microphone of the portable listening system. The mode input, Mode, is in an embodiment controlled by a voice detector, e.g. including or consisting of an own voice detector, which provides a control signal based on an analysis of the microphone signal (Ov). The delay unit d is adapted to provide a configurable delay controlled by input Del.

FIG. 2b shows an embodiment of the portable listening system of FIG. 2a, wherein the system comprises 2 separate physical bodies, a microphone device 1 comprising microphone 11, delay unit 12 and selector unit (SEL) and a listening device 2 comprising signal processor (DSP) and receiver 21. The two separate devices may be electrically connected, either by a wired connection or by a wireless connection. In an embodiment, a wireless link is setup by means of transceivers Tx and Rx in the microphone and listening devices, respectively. In FIG. 2b, the connection is indicated to be one-way from the microphone device 1 to the listening device 2 (transmission unit (Tx) in the microphone device 1 and reception unit (Rx) in the listening device 2). The link may alternatively be two-way (e.g. based on a plug and socket wired solution or a wireless connection. The wireless link is e.g. based on inductive communication or on radiated fields, e.g. analogue modulated link, e.g. an FM-link, or a digital link, according to a digital transmission standard, e.g. Bluetooth).

FIG. 2c shows another embodiment of the portable listening system of FIG. 2a, wherein the system is partitioned in at least 2 separate physical bodies. The microphone device 1 comprises microphone 11 adapted for picking up a users own voice and converting it to an own voice electrical signal OV′. The microphone device further comprises a transceiver Tx adapted for (at least) transmitting the own voice electrical signal Ov′ to the listening device. The listening device 2 comprises delay unit 12, selector unit (SEL), signal processor (DSP) and receiver 21. Additionally the listening device (e.g. a hearing instrument) comprises a microphone 23 (or a microphone system comprising a number of microphones) for converting an acoustic sound to an electrical input signal N-Ov. Preferably, the microphone 23 is specifically adapted for providing a representation of sounds in the environment that do NOT originate from the user's own voice. The listening device further comprises a transceiver Rx (at least) for receiving an audio signal AUX from an auxiliary audio source (i.e. in addition to or alternatively to the microphone input(s)). The auxiliary audio input can e.g. be the incoming part of a telephone conversation, or any other incoming audio signal, e.g. from an entertainment device. In this embodiment the selector unit (SEL) is adapted to select as an output one of four inputs, 1) AUX from the auxiliary audio source, 2) N-Ov from the microphone 23 of the listening device, 3) Ov from the microphone device and representing the user's own voice and 4) d-Ov from the delay unit (d) 22 representing a delayed version of the user's own voice. The selector is adapted to select one of the inputs based on a mode select input (Mode). The signal AUX is selected when the user wishes to listen to an audio signal from an auxiliary audio source (e.g. a music player or an incoming telephone call). The signal N-Ov from the microphone 23 of the listening device is selected, when the microphone device 1 is not used (e.g. when the listening device, e.g. a hearing instrument, has its normal function, where the microphone 23 is the source of acoustic input to the listening device). The Ov signal from the microphone device 1 representing an undelayed version of the user's own voice is selected when the microphone device is used as a pick up of the user's own voice (or any other voice or sound) without introducing additional delay. The d-Ov signal from the delay unit (d) 22 representing a delayed version of the user's own voice is selected when the portable listening system is in its anti-stuttering learning mode. The transceiver Rx of the listening device for receiving an auxiliary input may be adapted to support a one- or a two-way link to/from the auxiliary device (e.g. a mobile telephone or an audio selection device). In case the microphone device 1 receives an incoming telephone call and forwards it to the listening device, the incoming part of the conversation and the delayed or undelayed version of the user's own vice picked up by the microphone device may be multiplexed (e.g. added) and transmitted over the same link to the listening device. In that case the listening device only needs one transceiver (optionally comprising only a receiver, if a fixed transmission channel is established). Alternatively, such mixing may be performed in the listening device, e.g. by allowing a mixture (e.g. a SUM or a weighted SUM) of the delayed own voice (d-Ov) and the AUX (e.g. an incoming telephone signal) inputs to a selector/mixer (SEL) to be fed to the DSP.

In an embodiment, the selector unit in FIG. 2c is adapted to—at a given time—select one of the four inputs or to combine (e.g. add in an internal summation unit) two of more of the four inputs (e.g. AUX and N-Ov and (Ov OR d-Ov). This is preferable in an application where the microphone (system) 23 is adapted to pick up voices from the user's environment (preferably exclusive of the user's own voice) at the same time when an incoming telephone call is present at the AUX-input AND the user's own voice is picked up by the microphone device (directly Ov or delayed d-Ov) together with that of the other voices (N-Ov) and where the combined signal is intended to be processed by the signal processor (DSP) and presented to the user via the receiver 21.

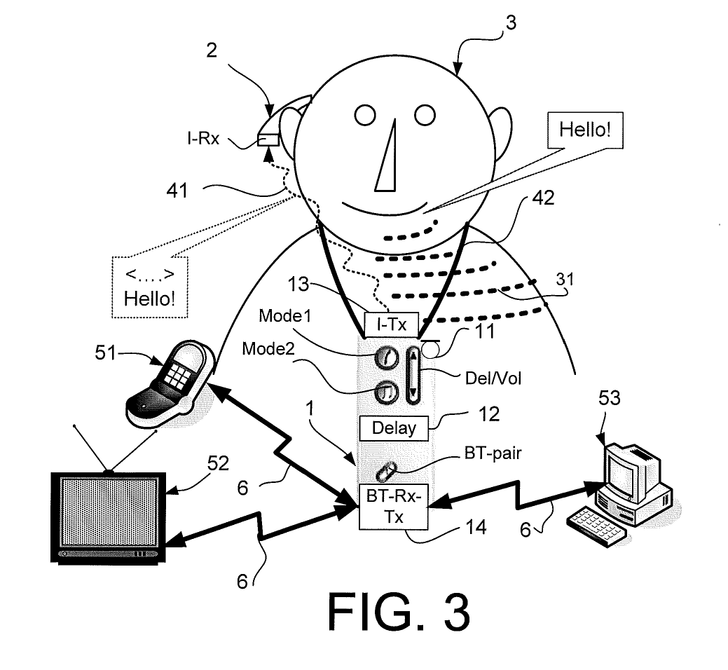

FIG. 3 shows an application scenario of an embodiment of a portable listening system according to the present application, wherein the microphone device 1 comprises an audio selection device adapted for receiving a multitude of audio signals (audio signals here shown from an entertainment device, e.g. a TV 52, a telephone apparatus, e.g. a mobile telephone 51 and a computer, e.g. a PC 53). In the embodiment of FIG. 3 the microphone device comprising a microphone 11 adapted for picking up the user's own voice is connected to the external audio sources 51, 52, 53 via wireless links 6, here in the form of digital transmission links according to the Bluetooth standard as indicated by the Bluetooth transceiver 14 (BT-Tx-Rx) in the microphone device 1. The audio sources and the microphone device may be paired using the button BT-pair. Once paired, the BT-address of the audio source may be stored in a memory of the microphone device for easy future pairing. The links may alternatively be implemented in any other convenient wireless and/or wired manner, and according to any appropriate transmission standard, possibly different for different audio sources. The microphone device 1 comprises a delay unit (Delay) for applying a configurable delay to the own voice electrical signal picked up by the microphone 11. The delay may be adjusted up and down in steps via a toggle button (Del/Vol), which at the same time may regulate volume of the audio signal transmitted to the listening device 2. Alternatively or additionally, the microphone device may comprise a display for displaying the current settings and/or for providing a user interface for entering and displaying parameter settings (e.g. delay, volume, program, etc.), e.g. in combination with the toggle button or via a touch screen capability or the like. The microphone device 1 further comprises a selector/combiner unit (not shown in FIG. 3) adapted for selecting and/or combining an appropriate signal or combination of signals for transmission to the listening device 2 as described in connection with FIG. 2c above. The intended mode of operation can be selected by the user via mode selection buttons Mode1 and Mode2. Here Mode1 indicates a telephone conversation mode (where the audio signal from a currently actively paired mobile telephone is selected) and Mode2 indicates an entertainment device mode (where the audio signal from a currently actively paired (or directly electrically connected) entertainment device is selected). The particular selected mode determines the signals to be selected/combined in the selector/combiner unit for transmission to the listening device. In Mode1, the own voice signal from microphone 11 AND the incoming signal from the mobile telephone is combined and transmitted to the listening device. In Mode 2, the audio signal from the selected entertainment device is selected and transmitted to the listening device. In a ‘normal mode’ where neither a telephone call nor an auxiliary audio signal from an entertainment device is present, the own voice signal from the microphone 11 of the microphone device can be activated and transmitted to the listening device with or without additional delay at the wish of the user.

The listening device 2 is shown as a device mounted at the ear of a user 3. The listening device 2 of the embodiment of FIG. 3 comprises a wireless transceiver, here indicated to be based on inductive communication (I-Rx). The transceiver (at least) comprises an inductive receiver (i.e. an inductive coil, which is inductively coupled to a corresponding coil in a transceiver (I-Tx) of the microphone device 1), which is adapted to receive the audio signal from the microphone device (either as a baseband signal or as a modulated (analogue or digital) signal, and in the latter case to extract the audio signal from the modulated signal). The inductive link 41 between the microphone device and the listening device is indicated to be one-way, but may alternatively be two-way (e.g. to be able to exchange control signals between transmitting 1 and receiving 2 device, e.g. to agree on an appropriate transmission channel).

The microphone device 1 is shown to be carried around the neck of the user 3 in a neck-strap 42. The neck-strap 42 may have the combined function of a carrying strap and a loop antenna into which the audio signal from the microphone device is fed for better inductive coupling to the inductive transceiver of the listening device.

The anti-stuttering learning mode is illustrated in FIG. 3 by the acoustic signal (‘Hello’) uttered by the user 3 in the form of acoustic waves 31 picked up by microphone 11 of the microphone device 1 and transmitted via the inductive link from the microphone device to the listening device (e.g. a hearing instrument or a headset) with a delay (‘< . . . > Hello!’). The delay may be adjusted by the user (according to needs, e.g. based on the advice of an audiologist or on a trial and error basis), e.g. in steps of 10 ms from 0 to 300 ms.

An audio selection device, which may be modified and used according to the present disclosure is e.g. described in EP 1 460 769 A1 and in EP 1 981 253 A1.

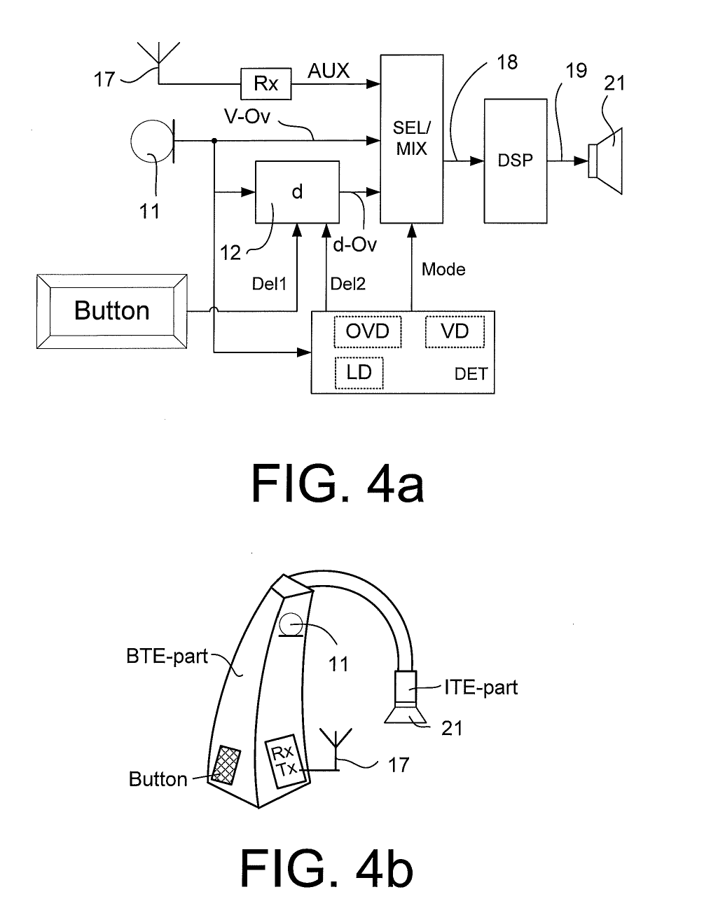

FIG. 4 shows an embodiment of a portable listening system comprising the functional components of the embodiment shown in FIG. 2a plus additional components. FIG. 4a illustrates the main functional components of the portable listening system. FIG. 4b illustrates such system embodied in a hearing instrument. The microphone (or microphone system) 11 is adapted to convert an input sound (e.g. comprising a person's voice) to an electrical signal V-Ov comprising a representation of a users own voice and/or other voices and/or other sounds, which is fed to a delay unit (d) 12 as well as to a selector/mixer unit (SEL/MIX). The delay unit 12 is adapted to delay the input signal V-Ov with an adjustable delay based on inputs Del1 and/or Del2 and to provide a delayed output signal d-Ov, which is fed to the selector/mixer unit (SEL/MIX). Additionally, the system comprises an auxiliary (direct) electric input from wireless transceiver circuitry comprising an antenna 17 and (at least) a wireless receiver Rx (possibly comprising appropriate demodulation circuitry to extract an audio signal from the received signal). The AUX signal from the wireless receiver Rx is fed to the selector/mixer unit (SEL/MIX). The AUX-signal is an audio signal and may e.g. comprise an input telephone signal, a sound signal from an entertainment device or the like. The selector/mixer unit is adapted to select one of its inputs AUX, V-Ov or d-Ov based on a mode input Mode and thus to provide a selected output signal 18 (being e.g. either the audio signal AUX, an undelayed signal from the microphone V-Ov or a delayed version d-Ov of the signal from the microphone), which (here) is fed to a processing unit (DSP) for further signal processing, e.g. for providing a frequency dependent gain, e.g. adapted to a user's needs. The processed output 19 of the signal processing unit (DSP) is fed to the receiver 21 for converting the processed output signal to an output sound for being presented to the user of the portable listening system. The output 18 from the selector/mixer unit (SEL/MIX) may alternatively be a mixture (e.g. a sum of, such as a weighted sum) of (some or all of) the inputs to the selector/mixer unit, e.g. the AUX- and the V-Ov- and/or the d-Ov-signals. The selector/mixer unit (SEL/MIX) is controlled by an input Mode from a detector unit (DET). The detector unit (DET) comprises one or more detectors for characterizing the microphone signal V-Ov. The detector unit (DET) comprises e.g. a voice detector (VD) for detecting the presence of a human voice in the input signal V-Ov at a given time and/or an own voice detector (OVD) for detecting the presence of a user's own voice in the input signal V-Ov at a given time and/or a level detector (LD) for detecting a level of the input signal V-Ov at a given time, e.g. to contribute to distinguishing between noise and speech (voice) signals. The configurable delay of the delay unit (d) is controlled by input signals Del1 and Del2. The control signal Del1 is generated by a user-operable activator (Button) of the system. The control signal Del2 is generated by the detector unit (DET), e.g. dependent on whether or not a human voice is detected in the input signal V-Ov, in particular dependent on whether or not a user's voice is detected. Preferably, the control signal Del1 from the user operable activator takes preference (overrides) the control signal Del2 from the detector unit (DET). The user-operable activator (Button) can in practice be implemented in any appropriate way, be it as an electromechanical activator, a command initiated from display, e.g. a touch screen, e.g. selected from a menu or an icon on the display. In a particular embodiment, the portable listening device shown in FIG. 4 is a hearing instrument adapted to a user's specific needs. In that case the user-operable activator for setting a delay of the delay unit preferably comprises an electromechanical activator, such as a push button or a wheel selector located on the hearing instrument. Alternatively the user operable activator can be implemented as part of a remote control for the hearing instrument. In the embodiment of FIG. 4, the microphone device of the portable listening device form part of the microphone system of the hearing instrument. In an embodiment, the hearing instrument is a BTE-type hearing instrument where a part of the ear worn part of the hearing instrument is adapted to be worn behind an ear of a user. Such embodiment is illustrated in FIG. 4b comprising a BTE-part and an ITE-part and a connecting element mechanically and electrically connecting the two parts of the hearing instrument. In the embodiment shown, the BTE-part comprises the microphone device 11 an activating element PB (at least for setting the delay of the delay unit (d in FIG. 4a)), denoted Button in FIG. 4a, and antenna 17 and transceiver circuitry RxTx for establishing a wireless link to another device, e.g. to another hearing instrument of a binaural hearing aid system and/or to a remote control for controlling (at least) some of the functionality of the hearing instrument (e.g. for setting a delay or choosing a hearing aid program for a particular acoustic environment), and/or to an audio gateway for receiving one of a multitude of audio signals and forwarding a selected one (e.g. from a telephone) to the hearing instrument). The ITE part comprises receiver 21. The rest of the components of FIG. 4a can be housed in either of the BTE- or the ITE-parts, but are typically embodied in the BTE-part. The connecting element is adapted to electrically connect the microphone device 11 to the receiver 21. Alternatively, the hearing instrument is an ITE-type hearing instrument, where the ear worn part is adapted to be located in an ear, e.g. an ear canal, of the user.

The invention is defined by the features of the independent claim(s). Preferred embodiments are defined in the dependent claims. Any reference numerals in the claims are intended to be non-limiting for their scope.

Some preferred embodiments have been shown in the foregoing, but it should be stressed that the invention is not limited to these, but may be embodied in other ways within the subject-matter defined in the following claims.

1. A portable listening system comprising a microphone device for picking up a person's voice and a listening device comprising an output transducer, the system being adapted to pick up the voice of a person wearing the portable system by the microphone device, converting the voice to an own voice electrical signal, to transmit the own voice electrical signal to the listening device, to receive the own voice electrical signal in the listening device, the system comprising a delay unit allowing to present the own voice electrical signal for the person wearing the portable system by the output transducer with a configurable delay.

2. A portable listening system according to claim 1 wherein the microphone device and the listening device constitute two physically separate bodies.

3. A portable listening system according to claim 1 wherein the listening device comprises a microphone for picking up an input sound.

4. A portable listening system according to claim 3 adapted to provide that the voice of a person with whom the wearer of the portable listening system communicates is picked up by the or a microphone of the listening device.

5. A portable listening system according to claim 1 adapted to provide that the transmission between the microphone device and the listening device is wired, e.g. via corresponding electrical connectors, or wireless, e.g. based on inductive communication.

6. A portable listening system according to claim 1 adapted to provide that delay is larger than 30 ms, such as in the range from 50 to 100 ms.

7. A portable listening system according to claim 1 adapted to provide that the listening device is a hearing instrument.

8. A portable listening system according to claim 1 adapted to provide that the delay is at least partially, such as that a major part of the delay is, implemented in the microphone device.

9. A portable listening system according to claim 1 adapted to provide that the microphone device comprises an audio selection device for selecting an audio signal from a multitude of audio signals and forwarding the selected audio signal to the listening device, optionally in combination with the voice signal picked up by the microphone device.

10. A portable listening system according to claim 9 adapted to provide that a selected audio signal can be a signal from a telephone.

11. A portable listening system according to claim 1 adapted to provide that the microphone device comprises a mobile telephone.

12. A portable listening system according to claim 10 adapted to provide that an audio signal representing an incoming signal from a telephone is forwarded to the listening device without additional delay.

13. A portable listening system according to claim 1 adapted to provide that a user can enable and/or disable the delay of the user's own voice.

14. A portable listening system according to claim 1 adapted to provide that the delay of the user's own voice is automatically enabled when a telephone conversation is initiated.

15. A portable listening system according to claim 1 adapted to provide that the microphone of the listening device can be manually or automatically disabled when a telephone conversation is initiated.

16. A portable listening system according to claim 1 comprising an own voice detector for detecting whether a given input sound originates from the voice of the user of the system.

17. A portable listening system according to claim 3 adapted to provide that the microphone of the listening system is dedicated for picking up another person's voice.

18. A portable listening system according to claim 1 wherein the microphone device form part of a microphone system of a hearing instrument.

19. A portable listening system according to claim 1 adapted to provide that the configurable delay can be set by a user continuously or in steps between a minimum value and a maximum value.

20. A method of using a portable listening system comprising a microphone device for picking up a person's voice and a listening device comprising an output transducer, the method comprising:
picking up the voice of a person wearing the portable system by the microphone device,
converting the voice to an own voice electrical signal,
transmitting the own voice electrical signal to the listening device,
receiving the own voice electrical signal in the listening device, and
delaying, with a configurable delay, the own voice electrical signal for the person wearing the portable system by the output transducer.
